(19) United States
(12) Patent Application Publication
(10) Pub. No.: US 2009/0069673 A1
(43) Pub. Date: Mar. 12, 2009

(54) SPINAL NEEDLE OPTICAL SENSOR

(75) Inventors: H. Charles Tapalian, Seekonk, MA (US); Francis J. Rogomentich, Wilmington, MA (US); Marc Steven Weinberg, Needham, MA (US)

Correspondence Address: FOLEY & LARDNER LLP, 111 HUNTINGTON AVENUE, 26TH FLOOR, BOSTON, MA, 02199-7610, US

(73) Assignee: The Charles Stark Draper Laboratory, Inc.

(21) Appl. No.: 12/049,692

(22) Filed: Mar. 17, 2008

Related U.S. Application Data
(60) Provisional application No. 60/895,252, filed on Mar. 16, 2007.

(51) Int. Cl. A61B 5/05

(52) U.S. Cl. 600/425

(57) ABSTRACT

An apparatus is disclosed including: an optical coherence tomographic system; a spinal needle having a needle tip adapted to penetrate tissue; and an optical delivery system adapted to direct probe light from the optical coherence tomographic system onto tissue located in front of the needle tip, collect test light backscattered from the tissue, and transmit the test light to the optical coherence tomographic system. The optical coherence tomographic system is adapted to provide information indicative of one or more properties of the tissue based on the test light.

### CROSS-REFERENCE TO RELATED PATENT APPLICATIONS

The present application claims benefit of U.S. Provisional Application Ser. No. 60/895,252 filed Mar. 16, 2007, the contents of which are incorporated by reference herein in their entirety.

### BACKGROUND

The present disclosure relates to medical sensors, for example, optical medical sensors.

Physicians use spinal needles for diagnostic, anesthetic, therapeutic and other procedures including, for example, lumbar punctures, spinal taps, and epidurals. For example, in a typical procedure a lumbar puncture is performed by inserting a needle in a patient's back, for example, at, or one space above or below, the L4-5 interspace. At this level, the dural sac contains cerebrospinal fluid and exiting nerve roots in the form of the cauda equina. In normal adults, the spinal cord itself has terminated at approximately at the tenth or eleventh thoracic vertebra. This is fortunate, since it keeps the spinal cord far from potential injury during a typical lumbar puncture. The patient is placed on his or her side and curled into a fetal position, with an attempt to keep the back perpendicular to the table, i.e., straight up and down.

A spinal needle inserted into the patient's back is typically guided to the dural sac crudely by touch and/or using external markers. For example, the top of the superior iliac crest is a good marker for the L4-5 interspace. The tester takes note of this space, and makes a thumbnail mark, or otherwise fixes the position of the L4-5 interspace. The spinal needle is inserted into the patient's back, e.g. halfway between the tips of the two spinous processes The needle is slowly advanced until pressure is felt. This pressure may represent bone. In this case, resistance to further needle advancement and patient discomfort will announce the presence of bone. When bone is encountered, the needle must be withdrawn almost to the skin for a re-approach with an adjusted angle.

Resistance is also felt when the posterior spinous ligaments and the dura are reached. In this case, a slight pop will be felt as these are penetrated and the needle is moved into its desired position. Correct positioning may be confirmed by noting fluid returned through a hollow channel in the needle. Once the needle is correctly positioned, it may be used to withdraw cerebrospinal fluid (CSF), check CSF pressure, introduce anesthesia or medication, etc. For example, in typical applications the spinal needle is hollow with the inserted needle tip cut at an angle to make a sharp point to enhance penetration. During insertion, a wire or cylinder fills the needle. The wire may have an angle identical to that of the needle and an index mated to the needle (so that the tapers of the needle and wire match). The wire prevents the needle from blockage by cut tissue during insertion. When the needle is in position, the wire is removed so that fluid can be drawn or inserted through the hollow channel.

Even under good conditions, techniques of the type described above may require repeated insertions and retractions of the spinal needle, and may result in repeated contact of the needle tip with bone, leading to substantial patient discomfort. A variety of conditions, including marked obesity, degenerative disease of the spine, previous spinal surgery, recent lumbar puncture, and dehydration, can make it difficult to perform lumbar punctures in the conventional manner.

A technique called video fluoroscopy is used when conventional lumbar puncture techniques are unsuccessful. Video fluoroscopy is a motion x-ray study of the bones and joints combining traditional fluoroscopy with the use of video technology to capture views of the neck (cervical spine) in motion. Video fluoroscopy requires a radiologist and a technologist to perform the procedure, thus making it prohibitively expensive for general use.

### SUMMARY OF THE INVENTION

The inventors have realized that by incorporating a spinal needle with an optical sensor (e.g. an optical tomographic system) capable of sensing the region located in front of the needle tip, a user may more easily and accurately guide the spinal needle to its desired location. For example, some embodiments permit doctors to sense the region in front of the needle tip (e.g. with >1 mm of axial visibility) and determine whether the needle is being directed toward bone (vertebrae) or the dura (the desired region). Such a device and technique helps doctors decrease the number of traumatic spinal taps (also referred to as lumbar punctures), reduces patient discomfort, and allows successful procedures under less than ideal conditions.

In one aspect, an apparatus is disclosed including: an optical coherence tomographic system; a spinal needle having a needle tip adapted to penetrate tissue; and an optical delivery system adapted to direct probe light from the optical coherence tomographic system onto tissue located in front of the needle tip, collect test light backscattered from the tissue, and transmit the test light to the optical coherence tomographic system. The optical coherence tomographic system is adapted to provide information indicative of one or more properties of the tissue based on the test light.

In some embodiments, the one or more properties of the tissue include reflectivity. In some embodiments, the information indicative of one or more properties of the tissue includes a depth-resolved reflectivity profile of the tissue located in front of the needle tip.

In some embodiments, the information indicative of one or more properties of the tissue includes information indicative of the presence or absence of a boundary between tissue of a first type and tissue of a second type located within the tissue located in front of the needle tip. In some embodiments, the boundary includes a boundary between soft tissue and bone.

In some embodiments, the optical coherence tomographic system includes one or more polarizing optical elements, and the information indicative of one or more properties of the tissue includes polarization resolved information. In some embodiments, the one or more properties of the tissue include birefringence.

In some embodiments, the spinal needle includes a hollow cavity extending from the needle tip to an end of the needle distal the needle tip. The optical delivery system includes an optical fiber extending from a first fiber end located proximal the needle tip, through the hollow cavity

Some embodiments including an optical connector adapted to optically connect a second end of the fiber to the optical tomographic system.

In some embodiments, the information indicative of one or more properties of the tissue includes a one dimensional depth-resolved profile of the tissue located in front of the needle tip.

In some embodiments, the optical fiber includes an optical fiber bundle having a plurality of fiber pixels, the fiber bundle extending from a first end located proximal the needle tip, through the hollow cavity, and terminating at a second end proximal to the optical coherence tomographic system.

In some embodiments, the second end of the fiber bundle includes a two dimensional array of fiber pixel faces, and where the optical tomographic system includes a scanning optical system adapted to direct the probe light onto selective ones of the fiber pixel faces. In some embodiments, the first end of the fiber bundle includes a two dimensional array of fiber pixel faces. In some such embodiments the apparatus also includes an imaging optical system positioned in front of the first end of the fiber bundle and adapted to image test light from an image plane located in front of the needle tip onto the two dimensional array of fiber pixel faces of the first end of the fiber bundle. In some embodiments, the imaging optical system includes an objective lens and a relay lens each positioned within the hollow cavity in front of the first end of the fiber bundle. In some embodiments, the imaging optical system includes a GRIN lens.

In some embodiments, the optical coherence tomography system is configured to: successively direct probe light onto each of the fiber pixel faces of the second end of the fiber bundle, for each successive fiber pixel face, determine a one dimensional depth-resolved profile of a corresponding portion the tissue located in front of the needle tip, and provide a two dimensional depth-resolved profile of the tissue located in front of the needle tip based on the one dimensional depth-resolved profiles. In some embodiments, the optical coherence tomography system is configured to generate a three dimension image based on the two dimensional depth-resolved profile.

In some embodiments, the fiber bundle includes about 10000 or more fiber pixels.

In some embodiments, the optical coherence tomographic system includes a time domain optical coherence tomography system.

In some embodiments, the optical coherence tomography system includes a spectral domain optical coherence tomography system.

In some embodiments, the optical coherence tomography system includes: a detector; an analyzer coupled to the detector; and an interferometer system. The interferometer system is configured to direct a probe light to the area of tissue located in front of the needle tip, collect test light from the area of tissue and combine the test light with reference light to interfere at the detector, the test and reference light having a common source; and vary an optical path length difference from the common source to the detector between interfering portions of the test and reference light. The detector is configured to produce an interference signal corresponding to an interference intensity measured by the detector as the optical path length difference is varied. The analyzer is configured to provide information indicative of one or more properties of the tissue based on the interference signal.

In some embodiments, the interferometer system is configured vary the optical path length difference from the common source to the detector between interfering portions of the test and reference light over a range larger than the coherence length of the common source.

In some embodiments, the interferometer system includes a movable optical element configured to vary the optical path length difference from the common source to the detector between interfering portions of the test and reference light.

In some embodiments, the common source includes a wavelength tunable source configured to vary the wavelength of the test and reference light to vary the optical path length difference from the common source to the detector between interfering portions of the test and reference light.

In some embodiments, the interference signal includes oscillations in response to the varying wavelength, and the analyzer is configured measure spectral oscillation components of the interference signal, and to provide information indicative of one or more properties of the tissue based on the measured spectral oscillation components.

In some embodiments, the optical delivery system is removably inserted into a hollow channel in the spinal needle, said hollow channel extending from the needle tip to an end of the needle distal said needle tip.

In another aspect, a method is disclosed including: providing a spinal needle sensor unit including: an optical coherence tomographic system; a spinal needle having a needle tip adapted to penetrate tissue; an optical delivery system adapted to direct probe light from the optical coherence tomographic system onto tissue located in front of the needle tip, collect test light backscattered by the tissue, and transmit the test light to the optical coherence tomographic system; where the optical coherence tomographic system is adapted to provide information indicative of one or more properties of the tissue based on the test light. The method further includes inserting the spinal needle into a subject having a spine; using the spinal needle sensor unit to determine information indicative of one or more properties of the tissue located in front of the needle tip; and guiding the spinal needle tip to a position proximal the spine based on the information indicative of one or more properties of the tissue located in front of the needle tip.

In some embodiments, using the spinal needle sensor unit to determine information indicative of one or more properties of the tissue located in front of the needle tip includes displaying an image representative of the tissue located in front of the needle. In some embodiments, the image is a three dimensional image.

In some embodiments, using the spinal needle sensor unit to determine information indicative of one or more properties of the tissue located in front of the needle tip includes determining information indicative of the presence of bone located in front of the needle tip.

In some embodiments, guiding the spinal needle tip to a position proximal the spine based on the information indicative of one or more properties of the tissue located in front of the needle tip includes avoiding contact of the needle tip to bone based on the information indicative of the presence of bone.

Various embodiments may include any of the above described features, alone or in combination.

### BRIEF DESCRIPTION OF THE DRAWINGS

FIG. 1 shows a spinal needle sensor and cross section of the spinal region of a patient.

FIG. 2A is a diagram of a portion of a spinal needle sensor.

FIG. 2B is a diagram of a portion of a spinal needle sensor.

FIG. 3 shows a depth profile obtained using spectral domain optical coherence tomography.

FIG. 4A illustrates the use of a spinal needle sensor in a lumbar puncture procedure.

FIG. 4B illustrates the use of a spinal needle sensor in a lumbar puncture procedure.

FIG. 5A is a diagram of a portion of an imaging spinal needle sensor.

FIG. 5B is a diagram of a fiber bundle.

FIG. 5C is a diagram of a portion of an imaging spinal needle sensor.

### DETAILED DESCRIPTION

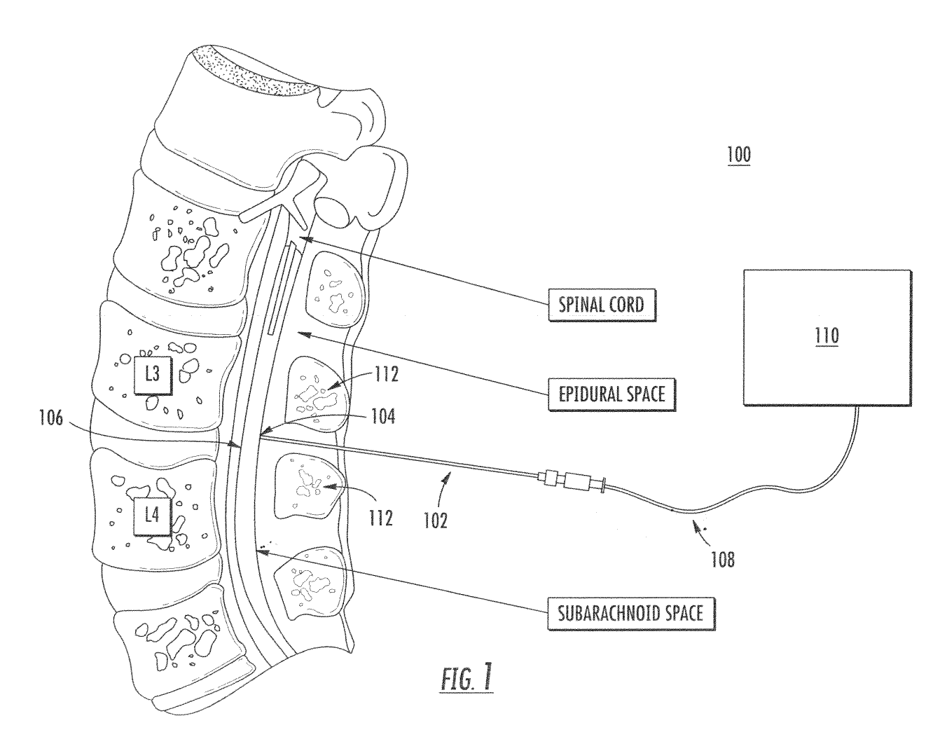

Referring to FIG. 1, spinal needle sensor 100 includes spinal needle 102. As shown, spinal needle 102 may be inserted through a patient's back, and needle tip 104 directed to a desired position, e.g., the spinal dura 106. Optical fiber 108 connects needle 102 to optical coherence tomographic (OCT) system 110. As described in detail herein, OCT system 110 optically senses the region in front of needle tip 104. This allows a user to more accurately insert needle 102 and position needle tip 104 in a desired position. For example, information provided by OCT system 110 may be used during insertion of needle 102 to avoid contact of needle tip 104 with areas of bone tissue 112.

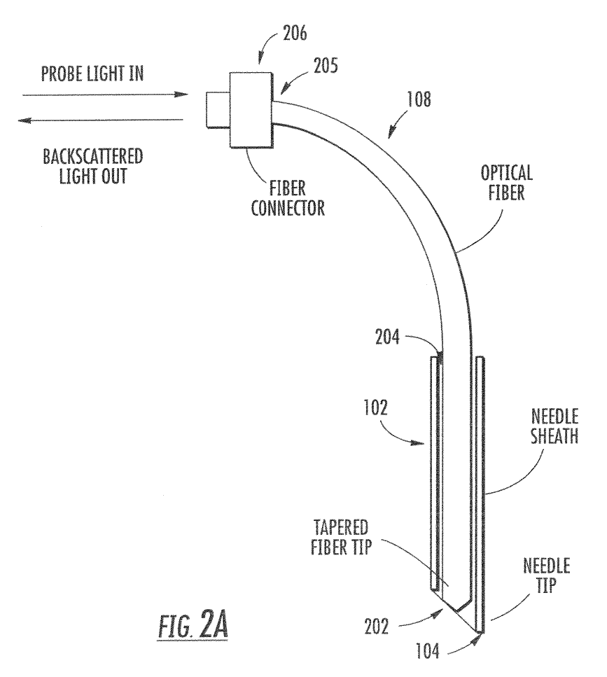

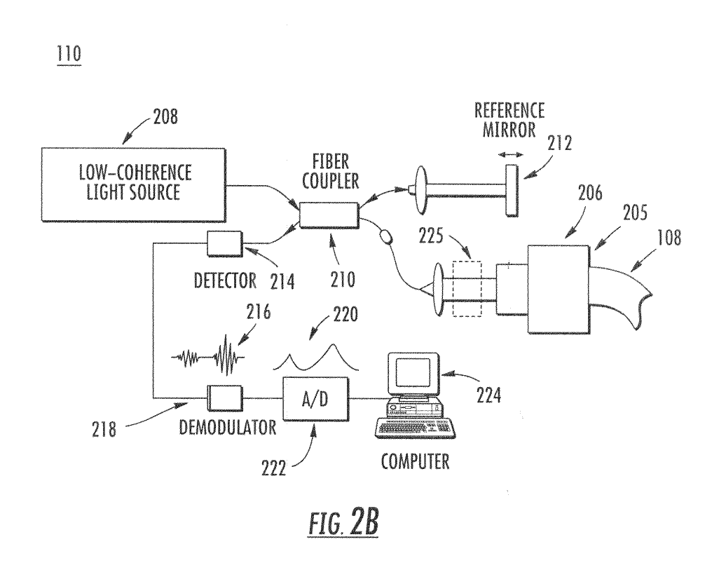

Referring to FIG. 2A, a single optical fiber 108 is used to transport the probe light from OCT system 110 (not shown) to needle tip 104 and then transport the light backscattered from tissue in front of needle tip 104 to the OCT system 110. Spinal needle 102 has a hollow cavity 204. Fiber 108 extends through cavity 204, such that needle 102 serves as a metal sheath around a portion of fiber 108. Fiber tip 202 (at the end of the needle) may utilize a conical taper in order to reduce the divergence of the probe light exiting from the needle. To match the needle's taper, the fiber could have a similar taper or the non fiber volume filled with material whose index of refraction matches or is close to that of human tissue.

In various embodiments, the fiber may be single mode or multimode. For typical applications, the size of fiber 108 is selected such that its diameter is as large as possible (determined by the diameter of cavity 204) in order to increase the amount of backscattered light collected. Fiber tip 205 (at the end of the fiber distal the needle) is received by fiber connector 206, which provides detachable optical coupling of fiber 108 to OCT system 110, e.g., as shown in FIG. 2B.

In some applications, optical fiber 108 may be removed from spinal needle 102 (e.g. after insertion). This allows, for example, spinal fluid to be withdrawn, or medicine, anesthesia, etc. delivered through spinal needle 102.

Note that the optical components enclosed in and/or attached to spinal needle 104 are preferably relatively simple and inexpensive. Accordingly, spinal needle 104 and enclosed/attached components may be made disposable, reducing or eliminating the need for repeated sterilization. In other embodiments, these components may be constructed from reusable (e.g. autoclavable) material.

Referring to FIG. 2B, OCT system 110 includes low coherence light source 208 optically coupled to Michelson interferometer 210. Probe light from the optical source 208 is split into 2 optical paths: a first directed through fiber 108 to the area of tissue located in front of needle tip 104 (not shown) and a second directed toward reference mirror 212. A portion of probe light traveling along the first path is backscattered (e.g. by reflection, refraction, diffraction or other optical process) from the tissue. The backscattered test light is combined on photodetector 214 with reference light reflected from reference mirror 212. Interference between the combined beams occurs only if the photons from both paths are coherent (i.e. the optical path length difference between the paths traveled by the test and reference light must be less than the coherence length of the probe light from source 208). In order to scan the axial (i.e. along the direction of the length of needle 102) depth of the sample, a variable optical delay may be introduced which scans (i.e. varies) the relative optical path lengths traveled by the test and reference light from common source 208. For example, as shown, reference mirror 212 is mounted on a translation stage which allows the position of the mirror to be varied to adjust the optical path length of the reference leg of interferometer 210.

Detector 214 measures, in response to the scan, interference intensity signal 216. When the relative optical path lengths are scanned over a range comparable to or greater than the coherence length of the probe light form source 208, signal 216 will exhibit areas of localized interference fringes at scan positions where the optical path length traveled by the test and reference light are equal.

Signal 216 is demodulated by demodulator 218, to provide fringe contrast signal 220. Fringe contrast signal 220 is converted to a digital signal by analog to digital converter 222, and passed to computer 224. As described in more detail below, computer 224 operates to analyze the fringe contrast signal using one or more of the many techniques known in the art, e.g. to provide a depth-resolved profile of the sample reflectivity (sometimes referred to as an A-scan). As described in detail below, such depth resolved information can be used to identify various features in the area of tissue in front of needle tip 104, such as interfaces between different tissue types (e.g. a bone/soft tissue interface).

In some embodiments, OCT system 110 can also incorporate additional signal discriminators such as probe light polarization provided by one or more polarizing optical elements (e.g., shown as dashed block 225). Many biological tissues such as tendon, muscle, nerve, bone, cartilage, and teeth exhibit birefringence and will therefore provide an enhanced reflectance signature at the tissue boundaries. In some embodiments, a polarization scrambler located, e.g., at the input 206 could be used in conjunction with a scanning reference mirror in order to provide polarization-sensitive depth profiles. The doctor utilizing the system would observe a live readout of the depth profile indicating the relative amplitudes of the reflecting tissues. In various embodiments, other suitable polarization sensitive optical coherence tomography techniques know in the art may be used.

In the embodiment described above, OCT system 110 is an example of a time domain OCT (TDOCT) system, i.e. and OCT system which utilizes a broadband optical source such that the coherence length is very short. As noted above, this provides axial sectioning (i.e. depth-resolution) of the system. Axial resolutions as high as 0.5 μm have been demonstrated using OCT.

In some embodiments, OCT system 110 may instead be a spectral domain OCT (SDOCT) system. In such embodiments, source 208 is replaced by a rapidly wavelength tunable narrowband source (e.g. a wavelength tunable laser or a narrowband source frequency modulated using an acousto-optic or electro-optic modulator, etc.). Detector 218 measures an oscillatory interference signal in response to rapid wavelength tuning of the source. This signal is digitized and analyzed by computer 224 to measure the spectral components of the interference signal, e.g., at evenly spaced wavenumbers. In some embodiments, the analysis includes Fourier transforming the measured interference signal from a time domain to a conjugate spectral domain. This is an SDOCT approach and is frequently referred to in the art as either optical frequency domain reflectometry (OFDR), wavelength tuning interferometry (WTI), or optical frequency domain imaging (OFDI). The measured spectral components may be analyzed using any of a variety of techniques know in the area to determine information about the properties of the tissue located in front of needle tip 104. For example, in some embodiments, a depth resolved reflectance profile of the tissue may be obtained.

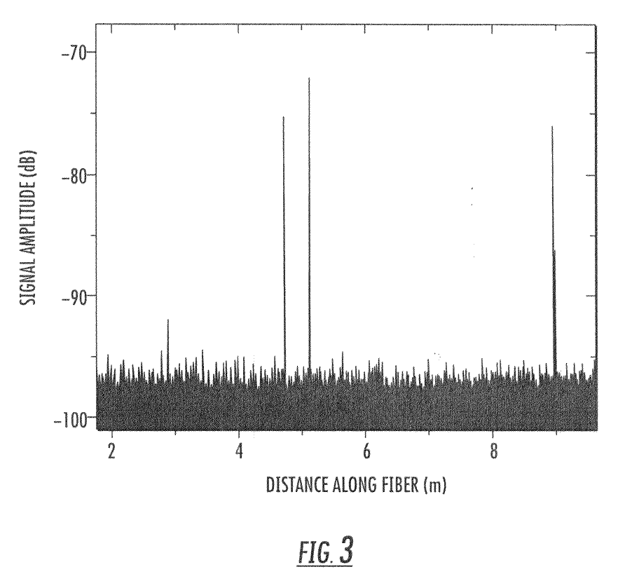

An example of a depth profile obtained using a fiber optic-based OFDR system is shown in FIG. 3. In this case, a depth profile of optical attenuation sources within a fiber-optic circuit was obtained using an optical frequency domain reflectometer (OFDR). Note that attenuations greater than 90 dB (reflection signal amplitudes less than −90 dB) can be resolved using this instrument. This technique may equally well be applied to detect, e.g. tissue type interfaces within the area of tissue in front of needle tip 104.

The maximum axial (i.e. depth) visibility which can be obtained using the techniques described herein is determined by a combination of system and biological parameters, including illumination power, probe fiber diameter, light divergence angle, the reduced scattering cross section of the “soft” tissue separating the back and spine, and the reflectivity of the soft/hard tissue interface. Conventionally available OCT systems typically provide only slightly greater than 1 mm of axial visibility in highly turbid media. However, in various embodiments, the range of axial visibility of the technique described herein may be greater due to, for example, the following factors. First conventional OCT systems acquire images with relatively high degrees of transverse and axial resolution (as high as 5 μm and 0.5 μm, respectively). This axial (depth) resolution is needed to reduce the unwanted out-of-focal-plane backscattered light; essentially sweeping away the fog that obscures the details of the image. The resolution requirements determine the signal-to-noise ratio (SNR) needed by the system which in turn determines the maximum axial visibility. Resolution requirements for the techniques described herein are typically substantially less than those required for conventional OCT imaging, thus resulting in a longer axial visibility distance. Second, the technique described herein will, in various embodiments, be used to determine the location of a soft/hard (bone) tissue interface. As the needle is moved across the region above the vertebrae, a large optical contrast in the depth profile will be detected due to the high diffuse reflectivity of the bone (at the interface). Since conventional OCT is used to acquire layered images within the same type of tissue (such as skin), the optical contrast and thus the SNR, would be lower than that for some embodiments of the technique at hand. The increased reflectivity at the interface therefore results in a greater SNR and a longer axial visibility distance.

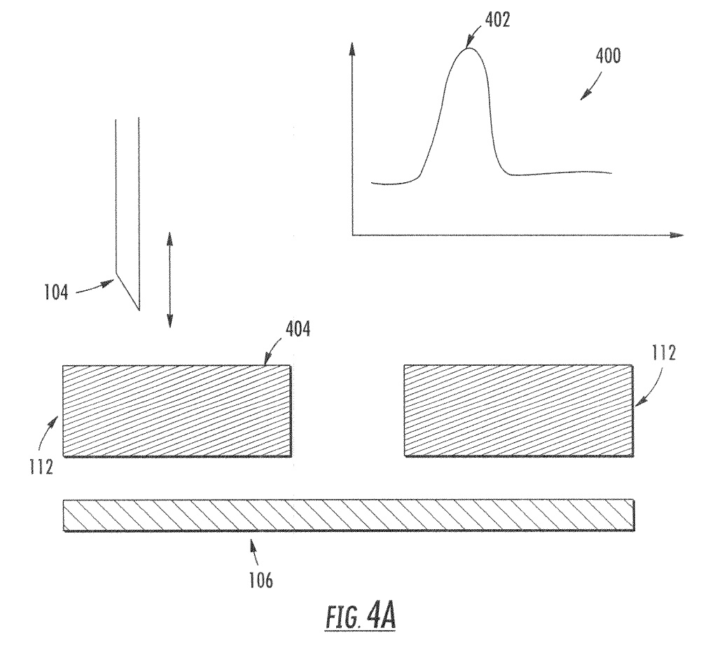

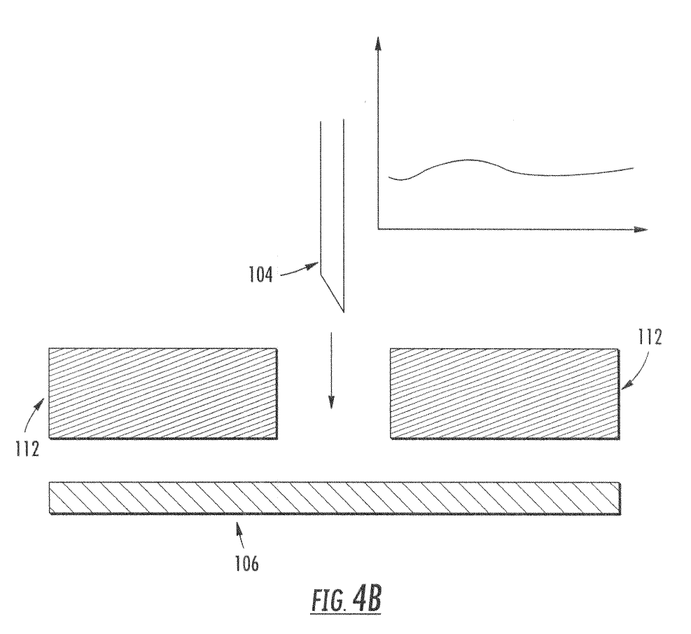

FIGS. 4A and 4B illustrate the use of spinal needle sensor 100 in a lumbar puncture procedure using the techniques described above. A user wishes to direct needle tip 104 into proximity or contact with dura 106, while avoiding bone tissue areas 112.

As illustrated in FIG. 4A, needle tip 104 is directed along a path which would bring it in contact with bone tissue 112. As needle tip 104 is advanced, depth resolved reflectance profile 400 (inset) of the tissue area in front of needle tip 104 is displayed to the user. As needle tip 104 approaches bone tissue 112, profile 400 exhibits reflectance peak 402, corresponding to the interface 404 between soft tissue and bone tissue 112. The user can therefore easily identify the presence of bone obstructing the path of needle tip 104 to dura 106, prior to contact of the tip to the bone obstruction.

Referring to FIG. 4B, the user has repositioned needle tip 104, such that the path of needle tip 104 is no longer obstructed by bone tissue 112. Therefore profile 400 no longer exhibits a reflectance peak corresponding to a bone/soft tissue interface. The user can therefore confirm that the path of needle tip 104 is free of obstruction and advance needle tip 104, allowing it to reach dura 106.

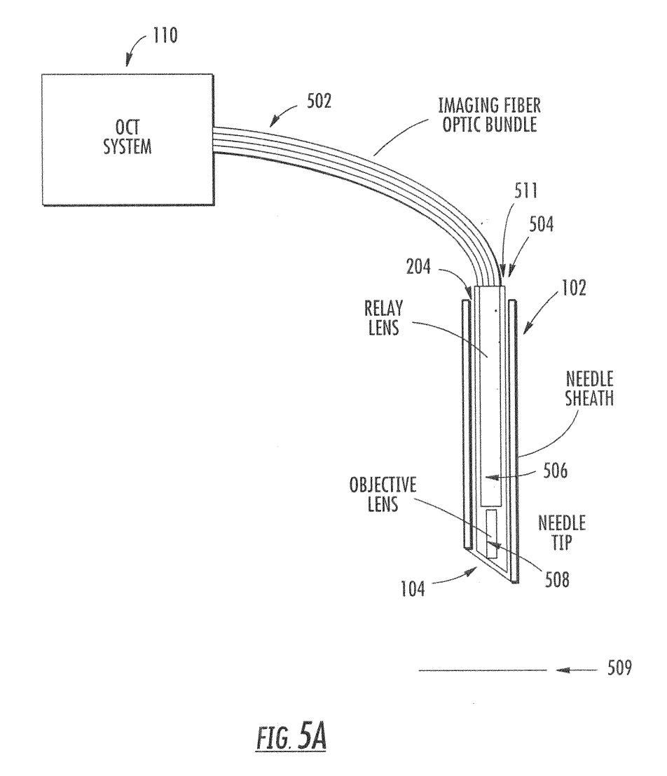

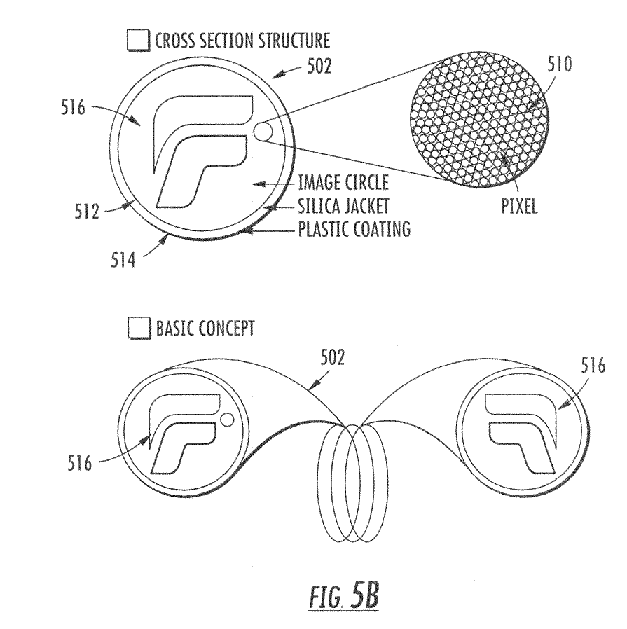

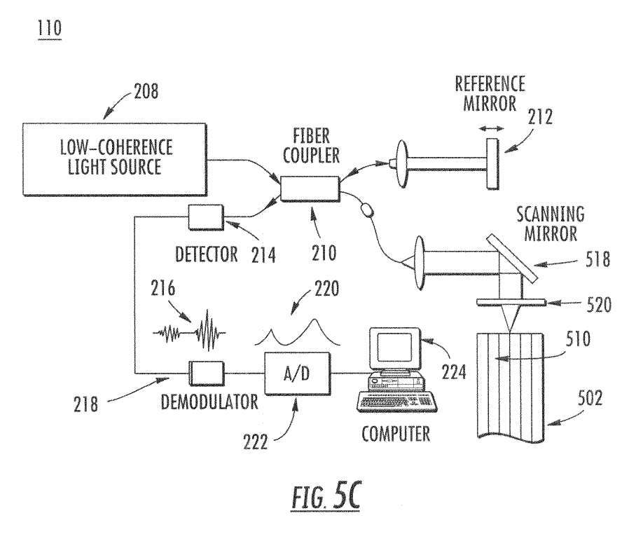

The examples described above feature systems which provide axial (i.e. depth) resolution of the features of the tissue area located in front of needle tip 104. However, some embodiments also provide transverse resolution. For example, as shown in FIG. 5A, fiber bundle 502 is used to transport probe light from OCT system 110 to needle 102 and then transport the light backscattered from the area of tissue located in front of needle tip 104 to OCT system 110. Lens system 504 is attached to the output end of the fiber bundle and positioned within cavity 204 of needle 102. In the embodiment shown, lens system includes 2 GRIN (gradient index) lenses, relay lens 506 and objective lens 508.

Lens system 504 images points on image plane 509 onto face 511 of fiber bundle 502. Referring to FIG. 5B, fiber bundle 502 includes multiple optical fiber pixels, e.g. pixel 510. The fiber pixels are contained by silica jacket 512 and plastic coating 514 Each end of fiber bundle 502 is a two dimensional array of fiber pixel faces. An image 516 projected on one end of the bundle (e.g. fiber bundle face 11) is relayed to the opposite end. In some embodiments, a coated fiber bundle structure consisting of 10000 fiber pixels is 450 μm or less in diameter.

Referring to FIG. 5C, OCT system 110 operates essentially as described above in reference to FIG. 2B. However, in order to obtain a 2-D (i.e. resolved in two dimensions transverse to needle 104) image, probe light is directed by scanning mirror 518 to lens 520. Lens 520 focuses the probe light onto a single fiber pixel (e.g. pixel 510 as shown) at input face 522 of fiber bundle 502. Fiber pixel 510 directs the probe light to a corresponding point on image plane 509, and returns backscattered test light. The backscattered test light is analyzed using the techniques described above to provide, for example, a depth resolved reflectance profile.

Scan mirror 520 then successively directs probe light to each of the remaining fiber pixels, and the process described above repeated for each fiber pixel to obtain a corresponding depth scan. The result is essentially a 2D array of depth scans. A 3-D image (i.e. both axially and transversely resolved may be generated from this array of depth scans).

Alternatively, scan mirror 520 may scan probe light over successive fiber pixels to provide a non-interferometric (i.e. fringe free) 2-D image. This image may be combined with one or more 1-D depth scans (e.g. corresponding to one or a few fiber pixels) to produce a 3-D image.

For some embodiments featuring imaging, the optical components enclosed by and/or attached to the needle may be relatively expensive. Therefore, in some embodiments, the needle and related parts may be constructed of reusable (e.g. autoclavable) materials.

Embodiments of the above described devices and techniques may be used in the setting where conventional lumbar punctures are performed (and by the same physicians). Such procedures will therefore be much less expensive than using fluoroscopy. Further, in some embodiments, some or all of the components of OCT system 110 may be enclosed in a single box and may, for example, be portable.

From the foregoing detailed description of the specific embodiments of the invention, it should be apparent that unique medical devices and medical kits have been described. Although particular embodiments have been disclosed herein in detail, this has been done by way of example for purposes of illustration only, and is not intended to be limiting with respect to the scope of the appended claims which follow. In particular, it is contemplated by the inventor that various substitutions, alterations, and modifications may be made to the invention without departing from the spirit and scope of the invention as defined by the claims. For instance and without limitation, the choice of needle gauge and fiber thickness, or wavelength of the illumination source used is believed to be matter of routine for a person of ordinary skill in the art with knowledge of the embodiments described herein.

Although the examples above feature a single detector to detect test and reference light, it is to be understood that, in some embodiments, multiple balanced detectors may be used.

Although the examples above feature a Michelson interferometer, is to be understood that any suitable interferometer configuration may be used including, e.g., Fizeau, Mach-Zehnder, or Twyman-Green.

Although the examples above feature optical coherence tomography, in some embodiments other optical sensing systems may be used to sense the properties of the area of tissue in front of a spinal needle tip. Examples of such optical sensing systems include confocal microscopy systems know in the art.

As used herein the term “light” is to be understood to include electromagnetic radiation both within and outside of the visible spectrum, including, for example, ultraviolet and infrared radiation.

One or more or any part thereof the techniques described above can be implemented in computer hardware or software, or a combination of both. The techniques can be implemented in computer programs using standard programming techniques following the method and figures described herein. Program code is applied to input data to perform the functions described herein and generate output information. The output information is applied to one or more output devices such as a display monitor. Each program may be implemented in a high level procedural or object oriented programming language to communicate with a computer system. However, the programs can be implemented in assembly or machine language, if desired. In any case, the language can be a compiled or interpreted language. Moreover, the program can run on dedicated integrated circuits preprogrammed for that purpose.

Each such computer program is preferably stored on a storage medium or device (e.g., ROM or magnetic diskette) readable by a general or special purpose programmable computer, for configuring and operating the computer when the storage media or device is read by the computer to perform the procedures described herein. The computer program can also reside in cache or main memory during program execution. The analysis method can also be implemented as a computer-readable storage medium, configured with a computer program, where the storage medium so configured causes a computer to operate in a specific and predefined manner to perform the functions described herein.

What is claimed is:

1. An apparatus comprising:
an optical coherence tomographic system;
a spinal needle having a needle tip adapted to penetrate tissue; and
an optical delivery system adapted to direct probe light from the optical coherence tomographic system onto tissue located in front of the needle tip, collect test light backscattered from the tissue, and transmit said test light to the optical coherence tomographic system;
wherein the optical coherence tomographic system is adapted to provide information indicative of one or more properties of the tissue based on the test light.

2. The apparatus of claim 1, wherein the one or more properties of the tissue comprise reflectivity.

3. The apparatus of claim 2, wherein information indicative of one or more properties of the tissue comprises a depth-resolved reflectivity profile of the tissue located in front of the needle tip.

4. The apparatus of claim 1, wherein the information indicative of one or more properties of the tissue comprises information indicative of the presence or absence of a boundary between tissue of a first type and tissue of a second type located within the tissue located in front of the needle tip.

5. The apparatus of claim 4, wherein the boundary comprises a boundary between soft tissue and bone.

6. The apparatus of claim 1, wherein the optical coherence tomographic system comprises one or more polarizing optical elements, and wherein the information indicative of one or more properties of the tissue comprises polarization resolved information.

7. The apparatus of claim 6, wherein the one or more properties of the tissue comprise birefringence.

8. The apparatus of claim 1,
wherein the spinal needle comprises a hollow cavity extending from the needle tip to an end of the needle distal the needle tip, and
wherein the optical delivery system comprises an optical fiber extending from a first fiber end located proximal the needle tip, through said hollow cavity

9. The apparatus of claim 8, further comprising an optical connector adapted to optically connect a second end of said fiber to the optical tomographic system.

10. The apparatus of claim 8, wherein the information indicative of one or more properties of the tissue comprises a one dimensional depth-resolved profile of the tissue located in front of the needle tip.

11. The apparatus of claim 1, wherein the optical fiber comprises an optical fiber bundle having a plurality of fiber pixels, said fiber bundle extending from a first end located proximal the needle tip, through said hollow cavity, and terminating at a second end proximal to the optical coherence tomographic system.

12. The apparatus of claim 11, wherein the second end of said fiber bundle comprises a two dimensional array of fiber pixel faces, and wherein the optical tomographic system comprises a scanning optical system adapted to direct the probe light onto selective ones of said fiber pixel faces.

13. The apparatus of claim 12, wherein the first end of said fiber bundle comprises a two dimensional array of fiber pixel faces, and further comprising:
an imaging optical system positioned in front of the first end of the fiber bundle and adapted to image test light from an image plane located in front of the needle tip onto the two dimensional array of fiber pixel faces of the first end of the fiber bundle.

14. The apparatus of claim 13, wherein the imaging optical system comprises an objective lens and a relay lens each positioned within the hollow cavity in front of the first end of the fiber bundle.

15. The apparatus of claim 14, wherein the imaging optical system comprises a GRIN lens.

16. The apparatus of claim 13, wherein the optical coherence tomography system is configured to:
successively direct probe light onto each of the fiber pixel faces of the second end of the fiber bundle,
for each successive fiber pixel face, determine a one dimensional depth-resolved profile of a corresponding portion the tissue located in front of the needle tip, and
provide a two dimensional depth-resolved profile of the tissue located in front of the needle tip based on the one dimensional depth-resolved profiles.

17. The apparatus of claim 16, wherein the optical coherence tomography system is configured to generate a three dimension image based on the two dimensional depth-resolved profile.

18. The apparatus of claim 11, wherein the fiber bundle comprises about 10000 or more fiber pixels.

19. The apparatus of claim 1, wherein the optical coherence tomographic system comprises a time domain optical coherence tomography system.

20. The apparatus of claim 1, wherein the optical coherence tomography system comprises a spectral domain optical coherence tomography system.

21. The apparatus of claim 1, wherein the optical coherence tomography system comprises:
a detector;
an analyzer coupled to the detector; and
an interferometer system configured to
direct a probe light to the area of tissue located in front of the needle tip,
collect test light from the area of tissue and combine the test light with reference light to interfere at the detector, said test and reference light having a common source; and
vary an optical path length difference from the common source to the detector between interfering portions of the test and reference light;
wherein the detector is configured to produce an interference signal corresponding to an interference intensity measured by the detector as the optical path length difference is varied;
wherein the analyzer is configured to provide information indicative of one or more properties of the tissue based on the interference signal.

22. The apparatus of claim 21, wherein the interferometer system is configured vary the optical path length difference from the common source to the detector between interfering portions of the test and reference light over a range larger than the coherence length of the common source.

23. The apparatus of claim 22, wherein the interferometer system comprises a movable optical element configured to vary the optical path length difference from the common source to the detector between interfering portions of the test and reference light.

24. The apparatus of claim 21, wherein the common source comprises a wavelength tunable source configured to vary the wavelength of the test and reference light to vary the optical path length difference from the common source to the detector between interfering portions of the test and reference light.

25. The apparatus of claim 25, wherein the interference signal comprises oscillations in response to the varying wavelength, and the analyzer is configured measure spectral oscillation components of the interference signal, and to provide information indicative of one or more properties of the tissue based on the measured spectral oscillation components.

26. The apparatus of claim 1, wherein the an optical delivery system is removably inserted into a hollow channel in the spinal needle, said hollow channel extending from the needle tip to an end of the needle distal said needle tip.

27. A method comprising:
providing a spinal needle sensor unit comprising:
an optical coherence tomographic system;
a spinal needle having a needle tip adapted to penetrate tissue;
an optical delivery system adapted to direct probe light from the optical coherence tomographic system onto tissue located in front of the needle tip, collect test light backscattered by the tissue, and transmit said test light to the optical coherence tomographic system;
wherein the optical coherence tomographic system is adapted to provide information indicative of one or more properties of the tissue based on the test light;
inserting the spinal needle into a subject having a spine;
using the spinal needle sensor unit to determine information indicative of one or more properties of the tissue located in front of the needle tip;
guiding the spinal needle tip to a position proximal the spine based on the information indicative of one or more properties of the tissue located in front of the needle tip.

28. The method of claim 27, wherein using the spinal needle sensor unit to determine information indicative of one or more properties of the tissue located in front of the needle tip comprises displaying an image representative of the tissue located in front of the needle.

29. The method of claim 27, wherein the image is a three dimensional image.

30. The method of claim 29, wherein using the spinal needle sensor unit to determine information indicative of one or more properties of the tissue located in front of the needle tip comprises determining information indicative of the presence of bone located in front of the needle tip.

31. The method of claim 30, wherein guiding the spinal needle tip to a position proximal the spine based on the information indicative of one or more properties of the tissue located in front of the needle tip comprises avoiding contact of the needle tip to bone based on the information indicative of the presence of bone.
